(19) United States
(12) Patent Application Publication
(10) Pub. No.: US 2006/0116648 A1
(43) Pub. Date: Jun. 1, 2006

(54) Port design and method of assembly

(76) Inventor: Bret Hamatake, Grantsville, UT (US)

Correspondence Address: MORRISON & FOERSTER, LLP, 555 WEST FIFTH STREET, SUITE 3500, LOS ANGELES, CA, 90013-1024, US

(21) Appl. No.: 11/189,577

(22) Filed: Jul. 26, 2005

Related U.S. Application Data
(60) Provisional application No. 60/591,387, filed on Jul. 26, 2004.

(51) Int. Cl. A61M 31/00

(52) U.S. Cl. 604/288.02

(57) ABSTRACT

An access port assembly having an ultrasonic weld energy director and method of assembling the access port utilizing the energy director. The access port may include a dual chamber port base and a port top for securing two septa on the base. An energy director may be positioned on the top end of the port base and a corresponding flat may be provided on the underside of the port top for receiving the energy director. Once the port top is aligned on top of the port based with the septa positioned in-between, far field welding may be implemented to connect the port top to the port base. The access port assembly may be further configured such that when the components are assembled for welding, the weld area is confined from air surrounding the port assembly.

### PRIORITY

This application claims the benefit under 35 U.S.C. § 119(e) of U.S. Provisional Application No. 60/591,387, filed Jul. 26, 2004, which is expressly incorporated by reference as if fully set forth herein.

### BACKGROUND

The present invention generally relates to a subcutaneously implantable access port. More specifically, the present invention relates to access port design and method for assembling an access port.

A variety of subcutaneously implantable access ports have been utilized by physicians to deliver fluids to, or withdraw fluids from, the bloodstream or other subcutaneous cavities inside a patient. One example of such an access port includes a needle-impenetrable housing, which encloses one or more fluid cavities and defines, for each of such fluid cavities, an access aperture communicating through the housing on the side thereof, which is adjacent to the skin of the patient where the access port is implanted into the body of a patient. A needle-penetrable septum is received in, and seals the access aperture. An exit passageway located in a port stem communicates with the fluid cavities for dispensing medication to a predetermined location in the body of the patient through an implanted catheter attached to the access port. Typically, the catheter is connected to the access port by placement of the proximal end of the catheter over the port stem. A locking sleeve or ring may be placed over the catheter at the proximal region of the catheter to secure the catheter on the port stem.

Examples of various access ports and catheter locking mechanisms are disclosed in U.S. Pat. No. 4,772,270, titled “INSEPARABLE PORT/CATHETER TUBE ASSEMBLY AND METHODS” issued to Wiita et al., dated Sep. 20, 1988; U.S. Pat. No. 5,632,729, titled “CATHETER CONNECTOR” issued to Cai et al., dated May 27, 1997; U.S. Pat. No. 4,929,236, titled “SNAP-LOCK FITTING CATHETER FOR AN IMPLANTABLE DEVICE” issued to Sampson, dated May, 29, 1990; U.S. Pat. No. 4,963,133, titled “CATHETER ATTACHMENT SYSTEM” issued to Whipple, dated Oct. 16, 1990; U.S. Pat. No. 5,045,060, titled “IMPLANTABLE INFUSION DEVICE” issued to Melsky et al., dated Sep. 3, 1991; U.S. Pat. No. 5,129,891, titled “CATHETER ATTACHMENT DEVICE” issued to Young, dated Jul. 14, 1992; U.S. Pat. No. 5,137,529, titled “INJECTION PORT” issued to Watson et al., dated Aug. 11, 1992; U.S. Pat. No. 5,312,337, titled “CATHETER ATTACHMENT DEVICE” issued to Flaherty et al., dated May, 17, 1994; U.S. Pat. No. 5,360,407, titled “IMPLANTABLE DUAL ACCESS PORT WITH TACTILE RIDGE FOR POSITION SENSING” issued to Leonard, dated Nov. 1, 1994; U.S. Pat. No. 5,399,168, titled “IMPLANTABLE PLURAL FLUID CAVITY PORT” issued to Wadsworth, Jr. et al., dated Mar. 21, 1995; U.S. Pat. No. 5,833,654, titled “LONGITUDINALLY ALIGNED DUAL RESERVOIR ACCESS PORT” issued to Powers et al., dated Nov. 10, 1998; U.S. Pat. No. 6,113,572, titled “MULTIPLE-TYPE CATHETER CONNECTION SYSTEMS” issued to Gailey et al., dated Sep. 5, 2000; each of which is incorporated herein by reference in its entirety.

Once the access port and the catheter have been implanted beneath the skin of a patient, quantities of medication or blood may be dispensed from the fluid cavity by means of a non-coring needle, which can be inserted into the fluid cavity through the skin of the patient and the penetrable septum. This medication may be directed to the distal end of the catheter to an entry point into the venous system of the body of the patient. Blood may also be withdrawn for sampling from the body of the patient through such an access port by applying negative pressure to the fluid cavity, which causes blood to be drawn through the catheter into the fluid cavity and then out of the body of the patient through the needle. To prevent clotting, thereafter, the withdrawal route may be flushed with a saline solution or heparin using a non-coring needle piercing the skin of the patient and the septum in the same manner as if a medication were being infused. Both intermittent and continual injections of medication may be dispensed by the access port. Continual access may involve the use of a non-coring needle attached to an ambulatory-type pump or gravity feed bag suspended above the patient. The ambulatory-type pump or the gravity feed bag continually delivers the medication or fluid through the needle to the fluid cavity in the access port and from there through the catheter to the entry point into the venous system.

A common method for assembling the plastic ports utilizes ultrasonic welding to connect the plastic parts. Typically, “shear joint” or “interference joint” is used as the connecting interface. The shear joint design is recommended by DuPont as the preferred joint design for crystalline plastic such as acetal resins (e.g., Delrin®). The shear joint design is described in detail on pages 100-103 of DuPont's General Design Principles for DuPont Engineering Polymers, Design Guide Module 1, Copyright © 2000, E.I. du Pont de Nemours and Company, which is incorporated herein by reference in its entirety. However, shear joint design generally requires precise features to be molded into the mating parts as well as precision alignment of those features along the entire length of the weld line. It is difficult to obtain closely matched mating features having a complex profile with injection molding. In particular, a typical dual port design requires a figure-8-shaped weld path. In addition, with the shear joint design it is difficult to provide welding between the septa in the access ports while maintaining a narrow profile between the septa. If the distance between the two septa is increased to accommodate a shear joint design, the overall dimensions of the port would also need to be increased.

Therefore, a connecting joint design with forgiving parts geometry, which requires less strict tolerances is desirable. Furthermore, a joint interface design that allows for welding to occur between the septa of the access port while maintaining a narrow profile between the septa may also provide added benefits.

### BRIEF SUMMARY

Accordingly, described herein are access ports which incorporate improved ultrasonic weld features. In one variation, the access port includes a dual port design with a built-in weld feature, which includes an energy director positioned on the top surface of the port base and a corresponding flat on the underside of the port top. Silicone rubber septa are captured between the port top and the port base during the ultrasonic welding process. Cylindrical features on the port top and the port base may provide general alignment during the assembly process. In this particular design, since the energy director on the base needs only to contact the flat surface on the top, precision alignment between the port top and port base is not required. Far field welding may then be utilized to weld the joint between the port top and the port base. Furthermore, since the weld occurs at the top of the port base adjacent the captured silicone septa, the potential strain or creep of the plastic features that compress the septa during and after assembly may be reduced by minimizing the amount of material between the weld joint and the load created by the compressed silicone rubber septa.

These and other embodiments, features and advantages of the present invention will become more apparent to those skilled in the art when taken with reference to the following more detailed description of the invention, in conjunction with the accompanying drawings that are first briefly described.

### BRIEF DESCRIPTION OF THE DRAWINGS

FIG. 1 illustrates one variation of a dual chamber access port.

FIG. 2 illustrates the dual chamber access port of FIG. 1 in a pre-assembled condition. As shown, the dual chamber access port includes a port top, two septa and a port base.

FIG. 3A illustrates a semi-assembled dual chamber access port prior to bounding of the port top to the port base through ultrasound welding.

FIG. 3B is an inset figure from FIG. 3A, showing an expanded view of the joint between the port top and the port base. An energy director is shown protruding from the port base and the port top sits on the tip of the energy director.

FIG. 4A illustrates the access port assembly of FIG. 3A after the parts are assembled through ultrasound welding of the port top to the port base. In the welding process the septa are secured between the port top and the port base.

FIG. 4B is an inset figure from FIG. 4A, showing an expanded view of the joint between the port top and the port base after the two parts have been welded together.

### DETAILED DESCRIPTION

The following detailed description should be read with reference to the drawings, in which like elements in different drawings may be identically numbered. The drawings, which are not necessarily to scale, depict selected preferred embodiments and are not intended to limit the scope of the invention. The detailed description illustrates, by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

The dual chamber access port is used herein as an example to illustrate the functionality of the different aspects of the invention disclosed herein. It will be understood that embodiments of the present invention may be applied in a variety of access ports (e.g., access ports with one, three, four or more fluid chambers), and need not be limited to the dual port design described herein. In addition, the invention may be adapted such that catheters having a plurality of lumens may be connected to access ports having one or more fluid chambers.

It must also be noted that, as used in this specification and the appended claims, the singular forms “a,” “an” and “the” include plural referents unless the context clearly dictates otherwise. Thus, for example, the term “a port” is intended to mean a single port or a combination of ports, “a fluid” is intended to mean one or more fluids, or a mixture thereof. In addition, it is to be understood, that unless otherwise indicated, this invention need not be limited to applications in human. As one of ordinary skill in the art would appreciate, variations of the invention may be applied to other mammals as well. Moreover, it should be understood that embodiments of the present invention may be applied in combination with various catheters, drug pumps, and infusion devices.

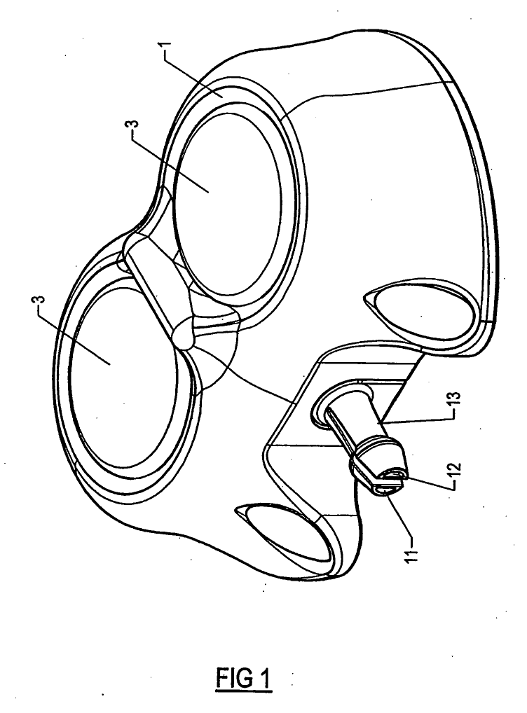

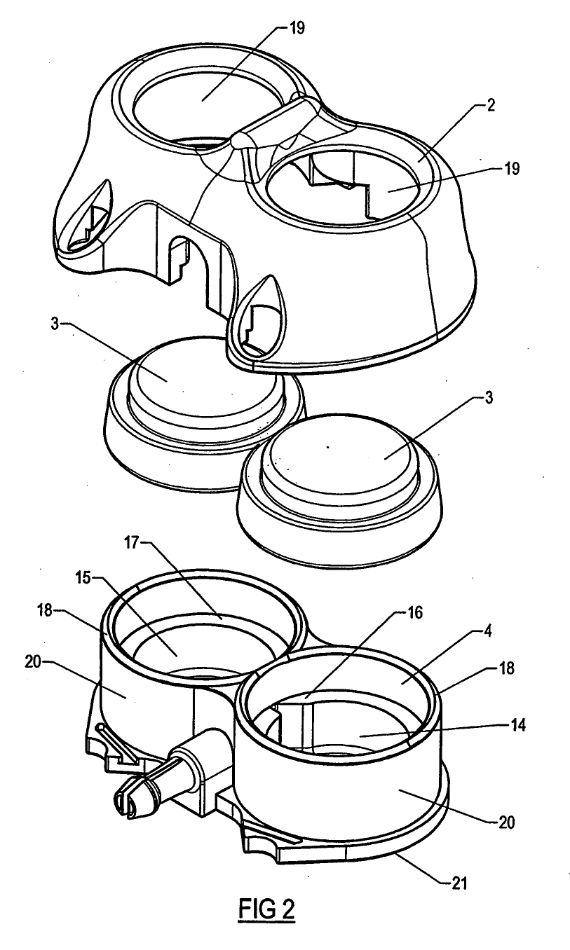

Referring to FIG. 1, one design variation of an access port 1 having dual chambers is shown in an assembled condition. Each of the two chambers is covered by a septum 3 for receiving a needle to infuse or withdraw fluids from the chamber. The outlets of the chambers connect to channels 11, 12 within a port stem 13, which allows fluids to flow in and out of the chambers. In this variation, the port stem 13 is part of the access port 1 and extends from the housing which supports the fluid chambers. As shown in FIG. 2, in this particular variation the access port includes three parts: (1) a port base 4 housing the two chambers 14, 15, where an upper portion 16, 17 of each of the chambers 14, 15 includes a receptacle for receiving a corresponding septum 3, (2) a pair of septa 3, and (3) a port top 2 for securing the two septa 3 within their corresponding receptacles in the chambers. In a preferred embodiment, the port base 4 and the port top 2 include a rigid amorphous plastic (e.g., Delrin®, Zytel®, Minlon®, Rynite® PET, etc.), and the septa 3 includes a silicone rubber.

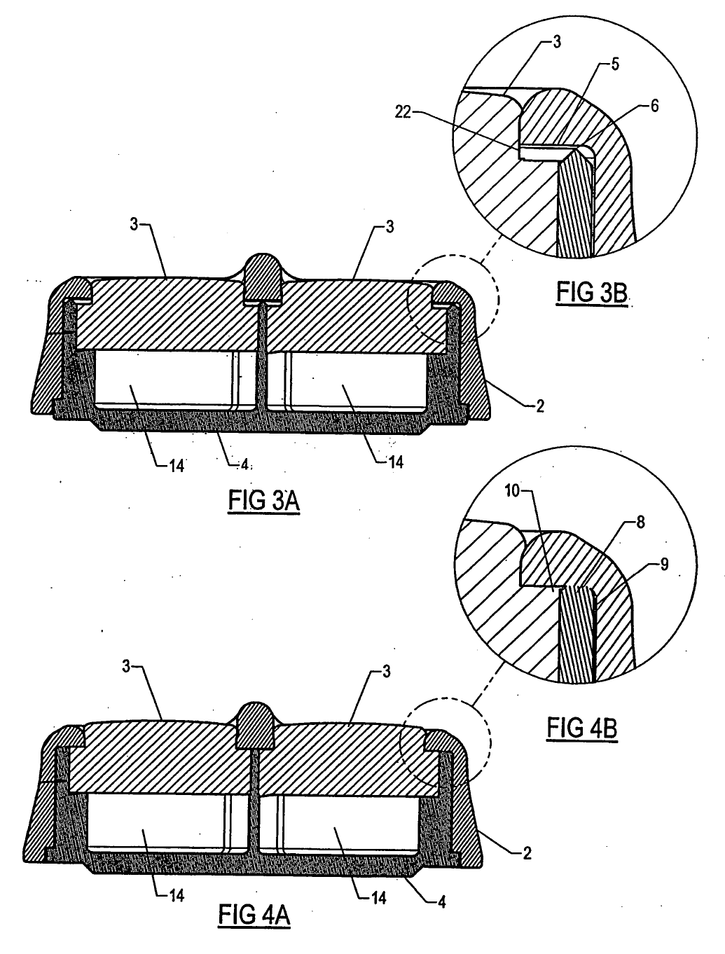

The primary interface between the port base 4 and the port top 2 is located at the top end 18 of the port base 4, which comes into direct contact with the under side 5 of the port top 2. This primary interface forms the welding joint. FIG. 3A illustrates the parts of the access port just before they are welded together. The weld features at the welding joint includes an energy director 6 on the port base 4 and a corresponding flat 5 on the underside of the port top 2. As shown in FIG. 3B, the port top is suspended on top of the energy director prior to the delivery of ultrasonic energy to weld the joint together. In this example, the energy director 6 includes a protrusion of a “V” shaped geometry. Other geometric profiles that are well known to one of ordinary skill in the art for directing ultrasonic energy for welding may also be utilized.

The cylindrical features on the inner surface 19 of the port top 2 and the corresponding outer surface 20 of the port base 4 provide general alignment during assembly. Since the energy director 6 on the port base 4 only needs to contact the flat surface 5 on the port top 2, precision alignment of the port top 2 and port base 4 is not required. In this variation, the weld occurs at the top of the port base adjacent to the captured silicone rubber septa 3, reducing the potential strain or creep of the plastic features that compress the septa during and after assembly by minimizing the amount of material between the welded joint and the load created by the compressed silicone rubber septa.

One approach for delivery of ultrasonic energy to the welding joint is through the use of a contoured welding horn that accommodates the variations in the molded surface of the bottom 21 of the port base 4, and a contour nest that matches the shape of the port top. Energy from the welding horn, in the form of vibrations oriented parallel to the long axis of the horn, has to transmit through the corresponding part to the area to be welded. Since the most efficient means for transmitting the vibrations into a part is on a path normal to the weld surface, in a port design having a flat bottom surface, it may be particularly desirable to transmit ultrasonic energy into the bottom of the port base. The port base can be easily coupled to the horn, with the flat bottom surface on the port base positioned normal to the horn axis to provide an efficient energy transfer interface.

In addition, it may be less desirable to transmit ultrasonic energy through the port top with a curved surface profile. Since the top surface of the port top has to accommodate the septum, it is generally designed with a curved surface profile. Because the curved surface of the port top is mostly not normal to the horn axis, it tends to poorly transmit the ultrasonic energy. Furthermore, much of that surface that is not normal to the horn axis tends to be scuffed by the vibrating motion of the horn, and the energy is converted to heat and scuffed plastic rather than being transmitted to the weld area.

Thus, preferably, the ultrasonic energy is delivered using far field welding. As the welding process is initiated, the energy director 6 on the port base 4 contacts the flat mating surface 5 of the port top 2. Energy from the welding horn is delivered onto the port base 4, and through the body of the port base 4 to the top 18 of the port base 4 where it is concentrated onto the tip of the energy director 6, which limits the initial contact with the flat mating surface 5 of the port top 2 to a very small area for rapid heating and melting. Once the narrow area defined by the energy director begins to soften and melt, impedance will drop and further melting occurs at a faster rate. The plastic in the energy director 6 melts first, and flows across the surface between the top of the port base 4 and the flat 5 on the under side of the port top 2. The edges 22 of the septa 3 create a confined space between the outside air and the weld area. This local confinement of the energy director prevents the outside air from prematurely cooling the welding interface and allows heat generated at the joint to be retained until the vibration ceases.

As the energy director 6 and the corresponding surface on the flat 5 melt, the port top 2 will collapse onto the port base 4, which eventually forms the assembled configuration shown in FIG. 4A. In addition, an optional gap 9 on the outside of the weld provides a flash overflow area, as shown in FIG. 4B. Since this flash overflow area is relatively small, the air within this area will not have a substantial effect on the welding process. The port top is welded onto the port base once the melted plastic at the interface between the port top and the port base solidifies.

An unexpected result of utilizing far field welding and an energy director at the top portion of the port base to concentrate the energy and form the welding interface, is that weld strength far exceeds the typical access port device requirements can be achieved. The strength of the joint is particularly surprising in view of the literature published by DuPont (the manufacture of Delrin® and other rigid amorphous plastics), which teaches away from the use of an energy director joint with far field welding (see pages 100-103 of DuPont's General Design Principles for DuPont Engineering Polymers, Design Guide Module 1, Copyright © 2000, E.I. du Pont de Nemours and Company). One of ordinary skill in the art would not have expected that such an approach would be capable of creating a quality joint on rigid amorphous plastic parts.

Furthermore, it is difficult to obtain closely matched mating features having a complex profile through injection molding, which is the preferred method of fabricating parts for an access port. Thus, for the traditional welding approach such as shear joint design, which requires precision alignment, the fabrication of a complex interface pattern may be difficult and costly. For example, it would be costly to manufacture corresponding port tops and port bases with a shear joint design for a dual port, which requires a figure-8 shaped weld path as shown on the top of the port base in FIG. 2. However, with the energy director design described herein, which allows for greater variations in part geometry, components for the access port may be manufactured at a lower cost. In addition, with multi-chamber ports it may be desirable to provide welding in-between the septa to improve the bond between the port top and the port base. The energy director design allows welding to occur between the septa while maintaining a narrow profile between the septa. This would be difficult to obtain with a shear joint design. One of ordinary skill in the art, having the benefit of the disclosure herein, would appreciate that the energy director design implemented with far field welding is also applicable in other multi-chamber port designs which may require complex circular welding paths. For example, a port with three or more chambers, and welding paths in-between the septa, may benefit significantly from the energy director design, which does not require precision alignment. In addition, one of ordinary skill in the art, having the benefit of this disclosure, would appreciate that the energy director weld joint design disclosed herein is also applicable in a single chamber port.

In yet another variation, the port top and the port base are configured to receive a compound septum, wherein two or more of the septum are provided as a continuous piece of silicone rubber (e.g., interlinks are provided between the septum). The welding interface and its corresponding energy director may be configured to loop around the compound septum.

This invention has been described and specific examples of the invention have been portrayed. While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited this specification are herein incorporated by reference in their entirety as if each individual publication or patent application were specifically and individually put forth herein.

What is claimed as new and desired to be protected by Letters Patent of the United States is:

1. An implantable access port assembly comprising:
a housing comprising a fluid chamber and a port stem extending from the housing, wherein the port stem includes an inner lumen forming a channel in fluid communication with the fluid chamber, the housing further comprising an energy director positioned on an upper surface of the housing for directing ultrasonic energy;
a cover with an access aperture for accessing the fluid chamber, wherein the cover comprises an inner surface configured for receiving the housing, and a flat on an under side of the cover for interfacing with the energy director; and
a septum configured to cover the fluid chamber and to be secured within the chamber by the cover.

2. The access port assembly according to claim 1, further configured such that when the housing, the cover and the septa are assembled together prior to delivery of ultrasonic energy for welding, a confined space is formed around the energy director.

3. The access port assembly according to claim 2, further configured such that when the housing, the cover and the septa are assembled together prior to delivery of ultrasonic energy for welding, a gap is provided between the housing and the cover to receive a flash overflow from the welding.

4. The access port assembly according to claim 2, wherein the energy director includes a V-shaped profile pointing upward from the housing.

5. The access port assembly according to claim 1, wherein the housing comprises a plurality of chambers, wherein the cover comprises a plurality of access apertures, and wherein the septum comprises a plurality of septa.

6. The access port assembly according to claim 5, further configured such that when the housing, the cover and the septa are assembled together prior to delivery of ultrasonic energy for welding, a confined space is formed around the energy director.

7. The access port assembly according to claim 6, wherein the energy director includes a V-shaped profile pointing upward from the housing.

8. The access port assembly according to claim 7, further configured such that when the housing, the cover and the septa are assembled together prior to delivery of ultrasonic energy for welding, a gap is provided between the housing and the cover to receive a flash overflow from the welding.

9. The access port assembly according to claim 5, wherein the energy director forms a continuous loop on the upper surface of the housing.

10. The access port assembly according to claim 1, wherein the housing comprises two chambers, the cover comprises two access apertures, and the septum comprises two septa.

11. The access port assembly according to claim 10, wherein the energy director forms a figure-8 pattern on the top surface of the housing.

12. The access port assembly according to claim 11, wherein the energy director includes a V-shaped profile pointing upward from the housing.

13. The access port assembly according to claim 12, further configured such that when the housing, the cover and the septa are assembled together prior to delivery of ultrasonic energy for welding, a confined space is formed around the energy director.

14. The access port assembly according to claim 13, further configured such that when the housing, the cover and the septa are assembled together prior to delivery of ultrasonic energy for welding, a gap is provided between the housing and the cover to receive a flash overflow from the welding.

15. A method of assembling an access port having a plurality of chambers, comprising the steps of:
placing a plurality of septa in a port base;
aligning a port top on the port base and capturing the septa in between the port top and the port base; and
directing ultrasonic energy into a bottom of the port base, allowing the ultrasonic energy to propagate through a body of the port base toward a top end of the port base, and concentrating the ultrasonic energy through an energy director positioned on the top end of the port base.

16. The method according to claim 15, wherein the port top further comprises a flat, on an underside of the port top, and the directing ultrasonic energy step further comprises transferring at least part of the ultrasonic energy from the energy director onto the flat.

17. The method according to claim 15, further comprising the step of forming a confined space around the energy director prior to the directing ultrasonic energy step.

18. The method according to claim 15, further comprising the step of allowing a flash overflow to flow into a built-in space between the port top and the port base.

19. The method according to claim 15, wherein the access port comprises two chambers, and the energy director forms a figure-8 pattern on the top end of the port base.

20. The method according to claim 16, further comprising the step of forming a confined space around the energy director prior to the directing ultrasonic energy step.

21. The method according to claim 20, further comprising the step of allowing a flash overflow to flow into a built-in space between the port top and the port base.

22. The method according to claim 21, wherein the access port comprises two chambers, and the energy director forms a figure-8 pattern on the top end of the port base.

23. A method of fabricating an access port comprising the steps of:
providing a port base, wherein the port base comprises an energy director positioned on a top end of the port base;
providing a port top, wherein the port top comprises a corresponding flat on an underside of the port top for receiving the energy director; and
welding the port top to the port base through far field welding.

24. The method according to claim 23, further comprising the step of forming a confined space around the energy director prior to the welding step.

25. The method according to claim 24, further comprising the step of allowing a flash overflow to flow into a gap between the port top and the port base.
